(19) United States
(12) Patent Application Publication
(10) Pub. No.: US 2024/0270576 A1
(43) Pub. Date: Aug. 15, 2024

(54) HEXAGONAL BORON NITRIDE POWDER AND METHOD FOR PRODUCING SAME, AND COSMETIC AND METHOD FOR PRODUCING SAME

(71) Applicant: Denka company Limited, Tokyo (JP)

(72) Inventor: Ryuki MATSUI, Tokyo (JP)

(73) Assignee: Denka company Limited, Tokyo (JP)

(21) Appl. No.: 18/568,066

(22) PCT Filed: Jun. 16, 2021
(86) PCT No.: PCT/JP2021/022924

§ 371 Date: Dec. 7, 2023

(51) Int. Cl. C01B 21/064; A61K 8/19; A61Q 19/00

(52) U.S. Cl. CPC C01B 21/0648; A61K 8/19; A61Q 19/00; A61K 2800/10; A61K 2800/651; C01P 2002/76; C01P 2004/61; C01P 2006/12

(57) ABSTRACT

A hexagonal boron nitride powder includes: secondary particles formed through agglomeration of primary particles of hexagonal boron nitride, in which a ratio of D50 to a BET specific surface area of the hexagonal boron nitride powder is 5 [μg/m] or more when D50 is defined as a particle size at which a cumulative value from smaller particle sizes reaches 50% of the total in a cumulative distribution of volume-based particle sizes measured through a laser diffraction/light scattering method.

### DESCRIPTION

### Technical Field

The present disclosure relates to a hexagonal boron nitride powder and a method for producing the same, and a cosmetic preparation and a method for producing the same.

### Background Art

Boron nitride has lubricity, high thermal conductivity, insulation properties, and the like, and is used in a wide range of applications, including solid lubricants, mold release agents, fillers for resins and rubber, raw materials for a cosmetic preparation (also called a cosmetic), and insulating sintered bodies with heat resistance.

Examples of functions of the hexagonal boron nitride powder mixed into a cosmetic preparation include improving slipperiness, spreadability, and concealability of the cosmetic preparation, and imparting glossiness to the cosmetic preparation. In particular, hexagonal boron nitride powders have superior slipperiness compared to a talc powder and a mica powder, which have similar functions, and are therefore widely used in the cosmetic preparation that requires excellent slipperiness. Patent Literature 1 proposes that the average particle size and the maximum particle size be within a predetermined numerical range to improve slipperiness of a hexagonal boron nitride powder.

### CITATION LIST

### Patent Literature

[Patent Literature 1] Japanese Unexamined Patent Publication No. 2018-165241

### SUMMARY OF INVENTION

### Technical Problem

To meet the increasingly high level of customer demands for a cosmetic preparation, there has been a demand for further improvement in the properties of raw materials used in the cosmetic preparation. For example, it is thought that it is necessary for raw materials used for foundation and the like to have superior spreadability. To improve spreadability, it is thought that it is effective to make the powder bulky to some extent.

The present disclosure provides a hexagonal boron nitride powder that can produce a cosmetic preparation with excellent spreadability, and a method for producing the same. In addition, the present disclosure provides a cosmetic preparation with spreadability using the above-described hexagonal boron nitride powder, and a method for producing the same.

### Solution to Problem

A hexagonal boron nitride powder according to an aspect of the present disclosure includes: secondary particles formed through agglomeration of primary particles of hexagonal boron nitride, in which a ratio of D50 to a BET specific surface area of the hexagonal boron nitride powder is 5 [μg/m] or more, when D50 is defined as a particle size at which a cumulative value from the smaller particle sizes reaches 50% of the total in a cumulative distribution of volume-based particle sizes measured through a laser diffraction/light scattering method.

The BET specific surface area in the above-described hexagonal boron nitride powder mainly depends on the particle size of the primary particles of the hexagonal boron nitride powder. On the other hand, D50 mainly depends on the particle size of the secondary particles formed through agglomeration of the primary particles. Accordingly, it can be stated that the ratio of D50 to the BET specific surface area correlates with the size of the secondary particles with respect to the primary particles and the proportion of the secondary particles in the above-described entire hexagonal boron nitride powder. Since the above-described ratio in the above-described hexagonal boron nitride powder is 5 [μg/m] or higher, the proportion of the secondary particles formed through agglomeration of the primary particles and/or the size of the secondary particles with respect to the primary particles are able to have been increased. The secondary particles have larger voids therein compared to the primary particles. Accordingly, a hexagonal boron nitride powder containing such secondary particles becomes bulky and has a fluffy appearance. The agglomerated secondary particles are spread while being destroyed when such a hexagonal boron nitride powder is spread. For this reason, the hexagonal boron nitride powder has excellent spreadability. Such a hexagonal boron nitride powder is suitable for use as a raw material for a cosmetic preparation.

The BET specific surface area of the above-described hexagonal boron nitride powder may be less than 3 [m²/g]. This makes it possible to increase the particle size of the primary particles and to sufficiently increase slipperiness.

D50 of the above-described hexagonal boron nitride powder may be 12 μm or more. Such a hexagonal boron nitride powder has superior spreadability.

The above-described hexagonal boron nitride powder may be used for a raw material for a cosmetic preparation. Since the above-described hexagonal boron nitride powder has excellent spreadability, it is suitable for use as the raw material for a cosmetic preparation.

A method for producing a hexagonal boron nitride powder according to an aspect of the present disclosure includes: a firing step of firing a mixed powder containing hexagonal boron nitride and an aid at 1600° C. or higher and lower than 1900° C. in an atmosphere of an inert gas, ammonia gas, or a mixed gas thereof to obtain a fired product containing hexagonal boron nitride having higher crystallinity than the hexagonal boron nitride of the mixed powder; a purification step of pulverizing, washing, and drying the fired product to obtain a dry powder; and an annealing step of annealing the dry powder at 1900° C. to 2100° C. in an atmosphere of an inert gas, ammonia gas, or a mixed gas thereof, in which, in the annealing step, the dry powder is heated at a temperature increase rate of 5° C./min or higher, and a time for heating to 1900° C. to 2100° C. is 2 hours or shorter.

In the above-described production method, the fired product containing hexagonal boron nitride having high crystallinity can be obtained by performing firing at the temperature of 1700° C. or higher and lower than 1900° C. using the aid. By pulverizing this fired product and then washing it, an amount of residual aid and the like can be reduced and grain growth during subsequent annealing can be suppressed. After drying, the fired product containing hexagonal boron nitride which has already been crystallized is annealed under predetermined conditions, which promotes formation of the secondary particles through agglomeration of the primary particles of the hexagonal boron nitride while inhibiting grain growth of the primary particles. Accordingly, the proportion of the secondary particles and/or the size of the secondary particles with respect to the primary particles are able to have been increased.

The secondary particles have larger voids therein compared to the primary particles. Accordingly, a hexagonal boron nitride powder containing such secondary particles becomes bulky and has a fluffy appearance. The agglomerated secondary particles are spread while being destroyed when such a hexagonal boron nitride powder is spread. Accordingly, according to the above-described production method, a hexagonal boron nitride powder having excellent spreadability can be produced. Such a hexagonal boron nitride powder is suitable for use as a raw material for a cosmetic preparation.

The above-described production method may further include, before the firing step: a calcination step of firing a raw material powder containing a boron-containing compound powder and a nitrogen-containing compound powder at 600° C. to 1300° C. in an atmosphere of an inert gas, ammonia gas, or a mixed gas thereof to obtain a calcined product containing low crystalline hexagonal boron nitride. The mixed powder in the firing step may contain the calcined product and the aid. In this manner, by performing calcination at a lower temperature than that in the firing step, grain growth is suppressed, and primary particles that are likely to form secondary particles contributing to improvement in spreadability are easily obtained.

A ratio of D50 to a BET specific surface area of the hexagonal boron nitride powder obtained in the annealing step of the above-described production method is 5 [μg/m] or more, when D50 is defined as a particle size at which a cumulative value from the smaller particle sizes reaches 50% of the total in a cumulative distribution of volume-based particle sizes measured through a laser diffraction/light scattering method.

A cosmetic preparation according to an aspect of the present disclosure includes the above-described hexagonal boron nitride powder. The above-described hexagonal boron nitride powder has excellent spreadability when spread. For this reason, the cosmetic preparation containing such hexagonal boron nitride powder have excellent spreadability.

A method for producing a cosmetic preparation according to an aspect of the present disclosure includes producing a cosmetic preparation using the hexagonal boron nitride powder obtained through any of the above-described production methods as a raw material. The hexagonal boron nitride powder obtained in the above-described production method has excellent spreadability when spread. For this reason, the cosmetic preparation produced using such a hexagonal boron nitride powder as a raw material have excellent spreadability.

### ADVANTAGEOUS EFFECTS OF INVENTION

According to the present disclosure, it is possible to provide a hexagonal boron nitride powder that can produce a cosmetic preparation with excellent spreadability, and a method for producing the same. In addition, according to the present disclosure, it is possible to provide a cosmetic preparation having excellent spreadability using the above-described hexagonal boron nitride powder, and a method for producing the same.

### DESCRIPTION OF EMBODIMENTS

Hereinafter, embodiments of the present disclosure will be described. However, the following embodiments are merely examples for describing the present disclosure and are not intended to limit the present disclosure to the following contents.

A hexagonal boron nitride powder according to one embodiment includes: secondary particles formed through agglomeration of primary particles of hexagonal boron nitride, in which a ratio of D50 to a BET specific surface area of the hexagonal boron nitride powder is 5 [μg/m] or more, when D50 is defined as a particle size at which a cumulative value from the smaller particle sizes reaches 50% of the total in a cumulative distribution of volume-based particle sizes measured through a laser diffraction/light scattering method. The ratio (D50/BET) may be 6 [μg/m] or more or 7 [μg/m] or more. This makes it possible to further increase the size and the proportion of the secondary particles and to further improve spreadability.

The ratio (D50/BET) may be less than 30 [μg/m] or less than 20 [μg/m]. This makes it possible to reduce roughness when the hexagonal boron nitride powder is used for a raw material for a cosmetic preparation. An example of a range of the above-described ratio (D50/BET) may be 5 [μg/m] or more and less than 30 [μg/m] or 7 [μg/m] or more and less than 20 [μg/m].

D50 in the present disclosure is measured using a commercially available laser diffraction particle size distribution measurement device. D50 may be 12 μm or more or 14 μm or more from the viewpoint of further improving slipperiness when the hexagonal boron nitride powder is used for a raw material for a cosmetic preparation. D50 may be 30 μm or less, 25 μm or less, or 20 μm or less from the viewpoint of reducing glare in appearance when the hexagonal boron nitride powder is used for a raw material for a cosmetic preparation. D50 can be adjusted by, for example, the particle size distribution of a raw material powder, the calcination temperature and time, the firing temperature and time, the annealing temperature and time, and the temperature increase rate. An example of a range of D50 may be 12 to 30 μm.

The BET specific surface area is a value measured using a commercially available specific surface area measurement device using nitrogen as an adsorption gas. The BET specific surface area may be less than 3 [m²/g] or less than 2.5 [m²/g]. This makes it possible to sufficiently increase not only spreadability but also slipperiness. The BET specific surface area may be 0.5 [m²/g] or more or 1 [m²/g] or more. This can improve adhesion to the skin and wrinkles. An example of a range of the BET specific surface area may be 0.5 to 3 [m²/g].

The bulk density of a hexagonal boron nitride powder may be 0.47 g/cm³ or less, 0.43 cm³ or less, and 0.37 cm3 or less. By having such a low bulk density, a hexagonal boron nitride powder having a fluffier appearance can be obtained. The bulk density can be measured in accordance with “Test methods for bulk density of fine ceramic powder” in JIS R1628-1997.

According to the present embodiment, the proportion of the secondary particles and/or the size of the secondary particles with respect to the primary particles in the hexagonal boron nitride powder are able to have been increased. The secondary particles can have larger voids therein compared to the primary particles. Accordingly, a hexagonal boron nitride powder containing such secondary particles becomes bulky and has a fluffy appearance. The agglomerated secondary particles are spread while being destroyed when such a hexagonal boron nitride powder is spread. For this reason, the hexagonal boron nitride powder has excellent spreadability. Such a hexagonal boron nitride powder is suitable for use as a raw material for a cosmetic preparation. That is, the present disclosure can also provide a method of using hexagonal boron nitride as a raw material for a cosmetic preparation.

A cosmetic preparation according to one embodiment contain the hexagonal boron nitride powder described above. Accordingly, the cosmetic preparation containing such a hexagonal boron nitride powder have excellent spreadability. Examples of the cosmetic preparation include foundation (powder foundation, liquid foundation, and cream foundation), face powder, point makeup, eye shadow, eyeliner, nail polish, lipstick, cheek rouge, and mascara. Among these, the hexagonal boron nitride powder is particularly well suited for foundation and eye shadow. The content of a hexagonal boron nitride powder in the cosmetic preparation is, for example, 0.1 to 70 mass %. The cosmetic preparation can be produced through a well-known method. A method for producing the cosmetic preparation includes, for example, a step of formulating and mixing a hexagonal boron nitride powder with other raw materials.

A method for producing a hexagonal boron nitride powder according to one embodiment includes: a calcination step of firing a raw material powder containing a boron-containing compound powder and a nitrogen-containing compound powder at 600° C. to 1300° C. in an atmosphere of an inert gas, ammonia gas, or a mixed gas thereof to obtain a calcined product containing hexagonal boron nitride; a firing step of firing a mixed powder containing the hexagonal boron nitride and an aid at 1600° C. or higher and lower than 1900° C. in an atmosphere of an inert gas, ammonia gas, or a mixed gas thereof to obtain a fired product containing hexagonal boron nitride having higher crystallinity than the hexagonal boron nitride of the mixed powder; a purification step of crushing, washing, and drying the fired product to obtain a dry powder; and an annealing step of annealing the dry powder at a temperature of 1900° C. to 2100° C. in an atmosphere of nitrogen gas, helium gas, argon gas, or the like.

Examples of boron-containing compounds include boric acid, boron oxide, and borax. Examples of nitrogen-containing compounds include cyandiamide, melamine, and urea. The molar ratio between boron atoms and nitrogen atoms in a raw material powder containing boron-containing compound powder and a nitrogen-containing compound powder may be boron atoms:nitrogen atoms=2:8 to 8:2, or 3:7 to 7:3. The raw material powder may contain components other than the above-described compounds. The raw material powder may contain carbonates such as lithium carbonate and sodium carbonate as a calcination aid. In addition, the raw material powder may contain a reducing substance such as carbon.

The raw material powder containing the above-described components is calcined with, for example, an electric furnace in an inert atmosphere such as nitrogen gas, helium gas, or argon gas, an ammonia atmosphere, or a mixed gas atmosphere in which these are mixed. The calcination temperature may be 600° C. to 1300° C., 800° C. to 1200° C., or 900° C. to 1100° C. The calcination time may be, for example, 0.5 to 5 hours or 1 to 4 hours.

The calcined product obtained through calcining contains at least one selected from the group consisting of low crystalline hexagonal boron nitride and amorphous hexagonal boron nitride. In the calcination step, the reaction of boron nitride is allowed to progress at a lower temperature than in the firing step to be described below. For this reason, grain growth can be suppressed, and the particle size of the primary particles of a boron nitride powder finally obtained can be reduced.

Next, the obtained calcined product is formulated and mixed with an aid to obtain a mixed powder. Examples of aids include borates such as sodium borate and carbonates such as sodium carbonate, calcium carbonate, and lithium carbonate. The formulation amount of the aid may be 2 to 20 parts by mass or 2 to 8 parts by mass based on 100 parts by mass of the calcined product containing hexagonal boron nitride. Such a mixed powder is fired in, for example, an electric furnace in an inert atmosphere such as nitrogen gas, helium gas, or argon gas, an ammonia atmosphere, or a mixed gas atmosphere in which these are mixed.

In the firing step, production and crystallization of boron nitride progress in the presence of the aid. As a result, the crystallinity of the boron nitride contained in the calcined product can be enhanced. The firing temperature is 1600° C. or higher and lower than 1900° C. This firing temperature may be 1650° C. to 1850° C. or 1650° C. to 1750° C. The firing time may be, for example, 0.5 to 5 hours or 1 to 4 hours.

If the firing temperature becomes too low, there is a trend for the secondary particles of the hexagonal boron nitride to be less likely to be produced sufficiently. If the size and/or proportion of the secondary particles decrease, there is a trend for slipperiness to decrease in a case where the hexagonal boron nitride is used for a raw material for a cosmetic preparation. The same trend is observed even when the firing time is too short. On the other hand, if the firing temperature is too high, the crystal growth and agglomeration of hexagonal boron nitride proceeds too much, which tends to cause strong glare when the hexagonal boron nitride is used for a raw material for a cosmetic preparation.

The fired product obtained in the firing step may contain impurities other than the hexagonal boron nitride. Examples of impurities include a residual aid and a water-soluble boron compound. In the purification step, such impurities are reduced through washing. After washing, solid-liquid separation and drying are performed to obtain a dry powder. Examples of washing liquids used for washing include water, an aqueous solution containing an acidic substance, an organic solvent, and a mixed liquid of an organic solvent and water. From the viewpoint of avoiding secondary contamination of impurities, water having an electric conductivity of 1 mS/m or less may be used. Examples of acidic substances include inorganic acids such as hydrochloric acid and nitric acid. Examples of organic solvents include water-soluble organic solvents such as methanol, ethanol, propanol, isopropyl alcohol, and acetone. The washing method is not particularly limited. For example, the fired product may be immersed in a washing liquid and stirred and washed, or the fired product may be washed by spraying a washing liquid.

After the completion of washing, the washing liquid may be subjected to solid-liquid separation through decantation or using a suction filter, a pressure filter, a rotary filter, a sedimentation separator, or a combination of these devices. A dry powder may be obtained by drying the separated solid content in a usual dryer. Examples of dryers include a shelf-type dryer, a fluidized bed dryer, a spray dryer, a rotary-type dryer, a belt-type dryer, and a combination thereof. After drying, for example, classification with a sieve may be performed to remove coarse particles. In the annealing step, the dry powder is heated at 1900° C. to

2100° C. with, for example, an electric furnace in an inert atmosphere such as nitrogen gas, helium gas, or argon gas, an ammonia atmosphere, or a mixed gas atmosphere in which these are mixed. This annealing temperature may be 1950° C. or higher from the viewpoint of sufficiently agglomerating the primary particles. In addition, the annealing temperature may be 2050° C. or lower from the viewpoint of suppressing grain growth of the primary particles. In the annealing step, since heating is performed at a temperature equivalent to that in the firing step, secondary particles in which primary particles are agglomerated can be sufficiently formed.

To suppress grain growth and excessive agglomeration of primary particles, the time for heating to a temperature of 1900° C. to 2100° C. in the annealing step may be 2 hours or shorter or 1 hour or shorter. On the other hand, from the viewpoint of forming sufficient secondary particles, the time for heating to a temperature of 1900° C. to 2100° C. in the annealing step may be 0.5 hours or longer.

In the annealing step, the temperature of the dry powder is increased as a temperature increase rate of 5° C./min or higher. By increasing the temperature at such a temperature increase rate, grain growth of primary particles and excessive agglomeration of primary particles can be suppressed. The temperature increase rate can be obtained by dividing the temperature difference (temperature increase range) between 1900° C. and a temperature at the start of temperature increase by a time required to reach 1900° C. from the start of temperature increase. The upper limit of the above-described temperature increase rate may be, for example, 15° C./min.

The above-described hexagonal boron nitride powder can be obtained in this manner. The description regarding the embodiment of the hexagonal boron nitride powder can be applied to the above-described production method. The method for producing a hexagonal boron nitride powder is not limited to the above-described embodiment. For example, the annealing step may be repeated plural times. In addition, after the annealing step, a crushing step of crushing a hexagonal boron nitride powder to the extent that secondary particles are not destroyed using a homogenizer applying ultrasound vibration may be performed. Some embodiments of the present disclosure have been described

above, but the present disclosure is not limited to any of the above-described embodiments.

### EXAMPLES

The contents of the present disclosure will be described in more detail with reference to examples and a comparative example, but the present disclosure is not limited to the following examples.

### Example 1

### Preparation of Hexagonal Boron Nitride Powder

### Calcination Step

100.0 g of a boric acid powder (purity of 99.8 mass % or more, manufactured by Kanto Chemical Co., Inc.) and 90.0 g of a melamine powder (purity of 99.0 mass % or more, manufactured by Wako Pure Chemical Industries, Ltd.) were mixed with each other with an alumina mortar for 10 minutes to obtain a mixed raw material. The mixed raw material after drying was placed in a container made of hexagonal boron nitride and placed in an electric furnace. The temperature was raised from room temperature to 1000° C. at a rate of 10° C./min while circulating nitrogen gas in the electric furnace. After holding the temperature at 1000° C. for 2 hours, the heating was stopped and the mixture was allowed to cool naturally. The electric furnace was open at a point in time when the temperature became 100° C. or lower. In this manner, a calcined product containing low crystalline hexagonal boron nitride was obtained.

### Firing Step

3.0 g of sodium carbonate (purity of 99.5 mass % or more) as an aid was added to 100.0 g of the calcined product and mixed with each other with an alumina mortar for 10 minutes. The mixture was placed in the above-described electric furnace. The temperature was raised from room temperature to 1700° C. at a rate of 10° C./min while circulating nitrogen gas in the electric furnace. After holding the temperature at a firing temperature of 1700° C. for 4 hours, the heating was stopped and the mixture was allowed to cool naturally. The electric furnace was open at a point in time when the temperature became 100° C. or lower. The obtained fired product was collected and pulverized with alumina mortar for 3 minutes to obtain a coarse powder of hexagonal boron nitride.

### Purification Step

In order to remove impurities contained in a coarse powder of hexagonal boron nitride, 30 g of the coarse powder was put into 500 g (concentration of nitric acid: 5 mass %) of dilute nitric acid and stirred at room temperature for 60 minutes. After stirring, solid-liquid separation was performed through suction filtration, and washing was performed by replacing the liquid with water (electric conductivity of 1 mS/m) until the filtrate became neutral. After washing, the solid was dried with a dryer at 120° C. for 3 hours to obtain a dry powder. Coarse grains were removed from the obtained dry powder using an ultrasonic vibrating sieve (KFS-1000, manufactured by Kowa Kogyosho Co., Ltd., mesh size of 250 μm).

### Annealing Step

The dry powder from which coarse grains were removed was placed in the above-described electric furnace. The temperature was raised from room temperature to 2000° C. at a rate of 5° C./min while circulating nitrogen gas in the electric furnace. After holding the temperature at 2000° C. for 2 hours, the heating was stopped and the mixture was allowed to cool naturally. The electric furnace was open at a point in time when the temperature became 100° C. or lower.

### Crushing Step

30 g of the coarse hexagonal boron nitride powder obtained was placed in 300 mL of water and subjected to ultrasonic dispersion using a homogenizer (manufactured by Sonic & Materials, Inc., trade name: VC505) under the conditions at 500 W and 20 kHz for 5 minutes. Thereafter, the dispersion was filtered to separate and dry solid contents. Coarse grains were removed from the obtained dry powder using an ultrasonic vibrating sieve (manufactured by Kowa Kogyosho Co., Ltd., KFS-1000, manufactured by Kowa Kogyosho Co., Ltd., mesh size of 250 μm) to obtain a hexagonal boron nitride powder of Example 1.

### Evaluation of Hexagonal Boron Nitride Powder

### Measurement of Particle Size Distribution

A volume-based particle size distribution of the hexagonal boron nitride powder prepared in Example 1 was measured using a laser diffraction particle size distribution measurement device (manufactured by Nikkiso Co., Ltd., device name: MT3300EX). In a cumulative distribution of volume-based particle sizes, a particle size (D50) when a cumulative value from small particle sizes reaches 50% of the total was obtained. The results are as shown in Table 2.

### Measurement of Specific Surface Area (N)

The BET specific surface area of the hexagonal boron nitride powder produced in Example 1 was measured through a BET 1-point method using a specific surface area measurement device (manufactured by Yuasa Ionics Co., Ltd., device name: MONOSORB). Nitrogen gas was used as an adsorption gas, and helium gas was used as a carrier gas. Measurement was performed after drying and degassing 1 g of a sample under the conditions of 300° C. for 15 minutes. The measurement results are as shown in Table 2. In addition, the ratio of D50 to the BET specific surface area is shown in the “D50/BET” column in Table 2.

Evaluation for spreadability

0.2 g of the hexagonal boron nitride powder was placed on one end of an artificial skin (length×width=10 mm×50 mm). The hexagonal boron nitride powder was spread along the surface of the artificial skin in the vertical direction using a spatula so that the hexagonal boron nitride powder was applied to the surface. Image analysis was performed using commercially available image analysis software (WinROOF) to determine the ratio of the coating area of the hexagonal boron nitride powder to the total area of the artificial skin. The larger this area proportion, the better the spreadability. The evaluation criteria for spreadability were as shown in Table 1 depending on the area proportion.

The evaluation results for spreadability are as shown in Table 2.

TABLE 1

| Area proportion | Determination |
|---|---|
| 95% or more | Very good |
| 80% or more and less than 95% | Good |
| 70% or more and less than 80% | Somewhat good |
| 60% or more and less than 70% | Fair |
| 40% or more and less than 60% | Poor |
| Less than 40% | Very poor |

### Example 2

A hexagonal boron nitride powder was prepared in the same manner as in Example 1 except that the heating time at 2000° C. in the annealing step was set to 1 hour. Then, various measurements and evaluations of the hexagonal boron nitride powder were performed in the same manner as in Example 1. The results are as shown in Table 2.

### Example 3

A hexagonal boron nitride powder was prepared in the same manner as in Example 1 except that the heating rate at 2000° C. from room temperature in the annealing step was set to 10° C./min. Then, various measurements and evaluations of the hexagonal boron nitride powder were performed in the same manner as in Example 1. The results are as shown in Table 2.

### Example 4

A hexagonal boron nitride powder was produced in the same manner as in Example 1 except that 3.0 g of sodium carbonate (purity of 99.5 mass% or higher) was added as an aid to a mixed raw material after drying and a firing step was performed without performing a calcination step. Then, various measurements and evaluations of the hexagonal boron nitride powder were performed in the same manner as in Example 1. The results are as shown in Table 2.

### Comparative Example 1

A dry powder obtained by removing coarse grains in a purification step without performing the annealing step in Example 1 was used as a hexagonal boron nitride powder of Comparative Example 1. Various measurements and evaluations of the hexagonal boron nitride powder were performed in the same manner as in Example 1. The results are as shown in Table 2.

### Comparative Example 2

A hexagonal boron nitride powder was prepared in the same manner as in Example 1 except that the heating rate at 2000° C. from room temperature in the annealing step was set to 2° C./min. Then, various measurements and evaluations of the hexagonal boron nitride powder were performed in the same manner as in Example 1. The results are as shown in Table 2.

TABLE 2

| | | BET specific | | |
|---|---|---|---|---|
| | D50 | surface area | D50/BET | |
| | (μm) | [m²/g] | [μg/m] | Spreadability |
| Example 1 | 20.8 | 2.7 | 7.7 | Very good |
| Example 2 | 25.9 | 2.9 | 8.9 | Very good |
| Example 3 | 14.8 | 2.3 | 6.4 | Good |
| Example 4 | 18.0 | 3.5 | 5.1 | Somewhat good |
| Comparative | 11.9 | 5.4 | 2.2 | Very bad |
| Example 1 | | | | |
| Comparative | 10.3 | 2.1 | 4.9 | Very bad |
| Example 2 | | | | |

Examples 1 to 4 all contained secondary particles in which primary particles were agglomerated. Examples 1 to 4 had a larger D50/BET value than Comparative Examples 1 and 2 and had a fluffy appearance. For this reason, Examples 1 to 4 contained more secondary particles contributing to improvement in spreadability than Comparative Examples 1 and 2 and had better spreadability. D50 of Example 1 was smaller than that of Example 2. This is thought to be due to the longer annealing time in Example 1, which caused primary particles to grow and agglomeration to break up. It is thought that if the annealing time is longer than that of Example 1, the influence on grain growth of the primary particles is eliminated, agglomeration proceeds, and D50 increases.

### INDUSTRIAL APPLICABILITY

According to the present disclosure, a hexagonal boron nitride powder that can produce cosmetics with excellent spreadability and a method for producing the same are provided. In addition, a cosmetic preparation with excellent spreadability using the above-described hexagonal boron nitride powder and a method for producing the same are provided.

1. A hexagonal boron nitride powder comprising:
secondary particles formed through agglomeration of primary particles of hexagonal boron nitride,
wherein a ratio of D50 to a BET specific surface area of the hexagonal boron nitride powder is 5 [μg/m] or more, and
wherein D50 is defined as a particle size at which a cumulative value from smaller particle sizes reaches 50% of the total in a cumulative distribution of volume-based particle sizes measured through a laser diffraction/light scattering method.

2. The hexagonal boron nitride powder according to claim 1,
wherein the BET specific surface area is less than 3 [m²/g].

3. The hexagonal boron nitride powder according to claim 1,
wherein D50 is 12 μm or more.

4. The hexagonal boron nitride powder according to claim 1, which is for a raw material for a cosmetic preparation.

5. A method for producing a hexagonal boron nitride powder, the method comprising:
a firing step of firing a mixed powder containing hexagonal boron nitride and an aid at 1600° C. or higher and lower than 1900° C. in an atmosphere of an inert gas, ammonia gas, or a mixed gas thereof to obtain a fired product containing hexagonal boron nitride having higher crystallinity than the hexagonal boron nitride of the mixed powder;
a purification step of pulverizing, washing, and drying the fired product to obtain a dry powder; and
an annealing step of annealing the dry powder at 1900° C. to 2100° C. in an atmosphere of an inert gas, ammonia gas, or a mixed gas thereof,
wherein, in the annealing step, the dry powder is heated at a temperature increase rate of 5° C./min or higher, and a time for heating to 1900° C. to 2100° C. is 2 hours or shorter.

6. The method for producing a hexagonal boron nitride powder according to claim 5, further comprising, before the firing step:
a calcination step of firing a raw material powder containing a boron-containing compound powder and a nitrogen-containing compound powder at 600° C. to 1300° C. in an atmosphere of an inert gas, ammonia gas, or a mixed gas thereof to obtain a calcined product containing hexagonal boron nitride,
wherein the mixed powder in the firing step contains the calcined product and the aid.

7. The method for producing a hexagonal boron nitride powder according to claim 5,
wherein a ratio of D50 to a BET specific surface area of the hexagonal boron nitride powder obtained in the annealing step is 5 [μg/m] or more, and
wherein D50 is defined as a particle size at which a cumulative value from smaller particle sizes reaches 50% of the total in a cumulative distribution of volume-based particle sizes measured through a laser diffraction/light scattering method.

8. A cosmetic preparation comprising:
the hexagonal boron nitride powder according to claim 1.

9. A method for producing a cosmetic preparation, the method comprising:
producing the cosmetic preparation using the hexagonal boron nitride powder obtained through the method for producing according to claim 5 as a raw material.
